(19) United States
(12) Patent Application Publication
(10) Pub. No.: US 2003/0032914 A1
(43) Pub. Date: Feb. 13, 2003

(54) Blood processing system

(75) Inventors: Masao Inoue, Kurashiki-shi (JP); Akihiro Ike, Tokyo (JP); Shuhei Nakaji, Kurashiki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: KURARAY CO. LTD., Kurashiki-shi (JP)

(21) Appl. No.: 10/214,201

(22) Filed: Aug. 8, 2002

(30) Foreign Application Priority Data
Aug. 10, 2001 (JP) 2001-243208

(51) Int. Cl. A61M 37/00

(52) U.S. Cl. 604/6.04

(57) ABSTRACT

To provide a blood processing system easy to operate and excellent in safety factor, the blood processing system includes a plasma separating unit for separating a plasma from a blood; a plasma purifying unit for purifying the separated plasma to which the plasma is introduced into the plasma purifying unit through a plasma introducing fluid circuit by means of a plasma feed pump, and a plasma return fluid circuit for returning the plasma, which has been purified by the plasma purifying unit, back to the plasma separating unit. A plasma inlet pressure gauge measures a pressure of the plasma at the plasma intake port of the plasma feed pump. A bypass fluid circuit extending between the plasma introducing fluid circuit and the plasma return fluid circuit for bypassing the plasma feed pump and the plasma purifying unit has a valve disposed therein for opening the bypass circuit. A control unit controls the plasma feed pump in reference to the plasma inlet pressure measured by the plasma inlet pressure gauge to render the plasma inlet pressure to fall within a predetermined range with respect to a preset pressure.

### BACKGROUND OF THE INVENTION

1. Field of the Invention

The present invention relates to a blood processing system including an extracorporeal circulating circuit having a plasma purifying unit for performing purification of a plasma that has been separated from patient's blood by a plasma separating unit and for returning the purified plasma to the patient with separated blood cell

2. Description of the Prior Art

The system has long been well known in which for purification of the blood, a plasma is separated from the blood by the use of a centrifugal separating unit and is subsequently purified by a plasma purifying unit before it is returned to the centrifugal separating unit. In this extracorporeal circulating system, a fluid circuit extending from the centrifugal separating unit to the plasma purifying unit and a fluid return circuit through which the purified plasma is returned to the centrifugal separating unit are provided with a plasma storage bag and a purified plasma reservoir, respectively A plasma feed pump is disposed downstream of the plasma storage bag and a purified plasma feed pump is also employed for feeding the purified plasma from the plasma purifying unit. A control unit is operatively associated with the plasma feed pump and the purified plasma feed pump so that the surface level of the plasma within the plasma storage bag as detected by a liquid level detector provided in the plasma storage bag can be kept within a predetermined range. See, for example, the Japanese Laid-open Patent Publications No. 60-256465 and No. 1-104272.

However, with the known blood processing system, the following problems have been found and are, hence, strongly desired to be resolved.

1) So long as the liquid surface level is detected within the plasma storage bag, no stable correlation between the liquid surface level and the actual amount of the plasma contained in the plasma storage bag cannot be secured because of deformation in shape of the plasma storage bag such as swelling and/or flattening. If as a result of change in shape of the plasma storage bag the surface level is erroneously detected, the plasma feed pump will be controlled irrespective of the actual amount of the plasma remaining within the plasma storage bag, resulting in unnecessarily excessive plasma processing. In such case, it is necessary to manually rectify the deformed shape of the plasma storage bag and/or the operating speed of the plasma feed pump and this is indeed cumbersome and time-consuming.

2) Even when as a result of occurrence of an abnormality during the blood purifying process the plasma feed pump is brought to a halt or is operated at a low flow rate, flow of the plasma from the centrifugal separating unit continues and, accordingly the amount of the plasma stored in the plasma storage bag increases and the plasma storage bag may rupture in the event of the worst case.

3) Considering that the plasma storage bag is generally used with no priming performed, there is a high possibility that residues brought about as a result of sterilization of the plasma storage bag may be dissolved into the plasma being processed.

### SUMMARY OF THE INVENTION

In view of the foregoing, the present invention has been devised to substantially eliminate the foregoing problems inherent in the conventional apparatus in which the plasma separated from the blood by the centrifugal separating unit is substantially purified and has for its object to provide a highly reliable and safe, high functional blood processing system that is effective to accomplish:

a) an improvement in operability achieved by the fact that an automatic control is performed to substantially equalize the flow rate of the separated plasma from the plasma separating unit to the flow rate of the plasma fed by the plasma feed pump;

b) an improvement in safety factor associated with the plasma purifying process in the event of an occurrence of abnormality; and

c) performance of the plasma purifying process with no plasma storage bag employed.

The inventors of the present invention have conducted a series of studies to achieve the foregoing objects and have found that those objects can be attained by designing the blood processing system in the following manner. Specifically, in the system for purifying the plasma separated from the blood by the plasma separating unit, the flow rate achieved by the plasma feed pump is automatically controlled to render the plasma inlet pressure, measured by a pressure gauge disposed in a plasma introducing fluid circuit, to fall within a predetermined range with respect to a preset pressure, so that the amount of the plasma to be supplied and the flow rate achieved by the plasma feed pump can be continuously controlled to coordinate with each other. Also, when as a result of occurrence of an abnormality during the blood purifying process the plasma feed pump is brought to a halt, the use is made of a fluid circuit for supplying the plasma from the plasma separating unit back to the plasma separating unit without the plasma being processed, so that any possible closure of the extracorporeal circulating circuit can be avoided Moreover, the use of the plasma storage bag which is considered an excessive use is eliminated because measurement of the surface level in the plasma storage bag hitherto done in the conventional system is superseded by pressure measurement in the extracorporeal circulation circuit.

More specifically, the present invention provides a blood processing system for purifying a plasma in a blood which includes a plasma separating unit for separating a plasma from a blood; a plasma purifying unit for purifying the separated plasma; a plasma introducing fluid circuit for introducing the separated plasma into the plasma purifying unit; a plasma feed pump disposed in the plasma introducing fluid circuit and having a plasma intake port and a plasma discharge port; a plasma inlet pressure gauge for measuring a pressure of the plasma at the plasma intake port of the plasma feed pump; a plasma return fluid circuit for returning the plasma, which has been purified by the plasma purifying unit, back to the plasma separating unit; a bypass fluid circuit extending between the plasma introducing fluid circuit and the plasma return fluid circuit for bypassing the plasma feed pump and the plasma purifying unit; a valve disposed in the bypass circuit for opening the bypass circuit in the event of an occurrence of operative abnormality in the blood processing system, and a control unit for controlling the plasma feed pump in reference to the plasma inlet pressure measured by the plasma inlet pressure gauge to render the plasma inlet pressure to fall within a predetermined range with respect to a preset pressure.

Thus, the present invention provides a highly reliable and safe, high functional blood processing system that is effective to accomplish an improvement in operability achieved by the fact that an automatic control is performed to substantially equalize the flow rate of the separated plasma from the plasma separating unit to the flow rate of the plasma fed by the plasma feed pump; an improvement in safety factor associated with the plasma purifying process in the event of an occurrence of abnormality; and performance of the plasma purifying process with no plasma storage bag employed.

In a preferred embodiment, the control unit has a function of controlling the plasma feed pump to render the plasma inlet pressure to fall within the range of, for example, ±5 mmHg with respect to the preset pressure.

In a preferred embodiment, the control unit has a function of controlling the plasma feed pump to render the flow of the plasma discharged from the plasma feed pump to be within a range of, for example, ±5 ml/min with respect to the flow of the plasma from the plasma separating unit.

The blood processing system of the present invention may further include a pressure gauge disposed between the plasma feed pump and the plasma purifying unit for detecting a plasma purifier pressure, in which case in the event that the plasma purifier pressure exceeds a predetermined value, the control unit halts the plasma feed pump and causes the valve on the bypass fluid circuit to open

In one preferred embodiment, the blood processing system of the present invention may also include a pressure gauge disposed in the plasma return fluid circuit for detecting a pressure of the plasma being returned, in which case in the event that the pressure of the plasma being returned exceeds a predetermined value the control unit halts the plasma feed pump and causes the valve on the bypass fluid circuit to open.

In a preferred embodiment of the present invention, in the event that the plasma feed pump is halted, the control unit may have a function of causing the valve on the bypass fluid circuit to open.

In another preferred embodiment, the plasma purifying unit may be selected from the group consisting of a plasma component fractionating membrane module and a plasma component adsorbent unit.

In a further preferred embodiment, the plasma purifying unit may include the plasma component fractionating membrane module. In such case, the blood processing system may furthermore include a filtrate feed pump operable in association with the plasma feed pump for draining a filtrate separated from the plasma by the plasma component fractionating membrane module.

In a still further preferred embodiment, where the plasma purifying unit may include the plasma component fractionating membrane module, the blood processing system of the present invention may also include a supplementary liquid supply circuit for supplying a supplementary liquid to the plasma component fractionating membrane module by means of the filtrate feed pump to thereby supplement a filtrated plasma with the supplementary liquid in a quantity corresponding to the quantity of the filtrate separated from the plasma.

### BRIEF DESCRIPTION OF THE DRAWINGS

In any event, the present invention will become more clearly understood from the following description of preferred embodiments thereof, when taken in conjunction with the accompanying drawings. However, the embodiments and the drawings are given only for the purpose of illustration and explanation, and are not to be taken as limiting the scope of the present invention in any way whatsoever, which scope is to be determined by the appended claims. In the accompanying drawings, like reference numerals are used to denote like parts throughout the several views, and:

FIG. 1 illustrates a diagram showing a fluid flow circuit of a plasma component adsorbing apparatus according to a preferred embodiment of the present invention; and

FIG. 2 illustrates a diagram showing the fluid flow circuit of the plasma component adsorbing apparatus according to another preferred embodiment of the present invention.

### DETAILED DESCRIPTION OF THE EMBODIMENTS

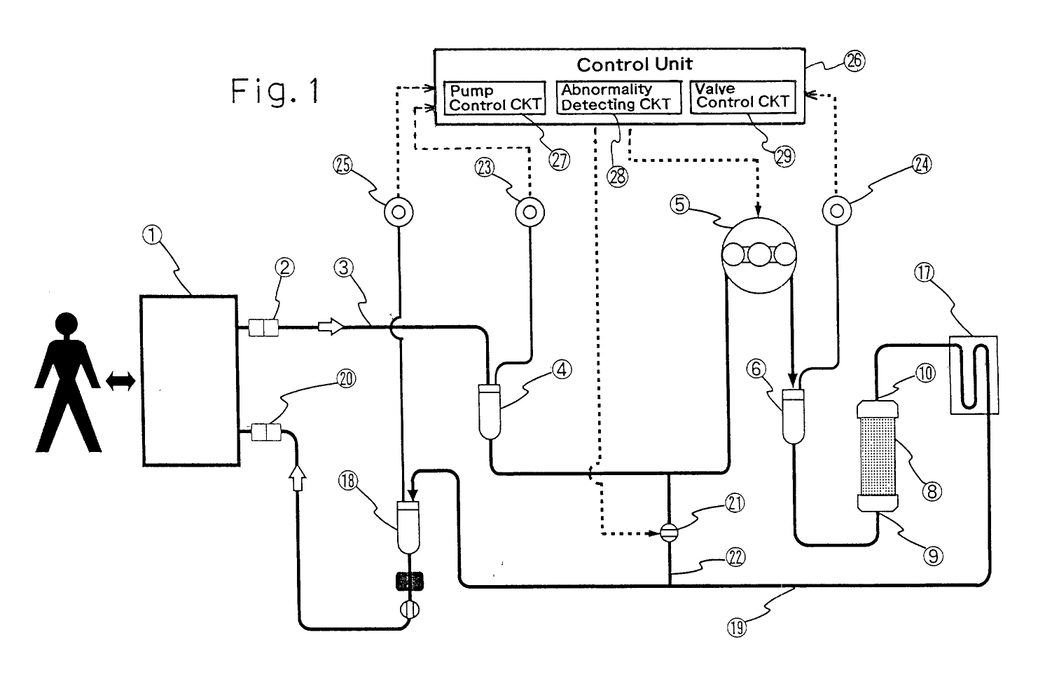

Referring first to FIG. 1 showing a blood processing system according to a preferred embodiment of the present invention. The illustrated blood processing system is a plasma component adsorbing system and includes a plasma separating unit 1 for separating blood, drawn from a patient to be treated, into a blood cell and a plasma and the plasma is supplied into a plasma introducing fluid circuit 3 through a plasma inlet 2. The plasma separating unit 1 may be, for example, a centrifugal separator such as “COBE Spectra”, “Baxter CS-3000 Plus”, or “HAEMONETICS MCS”, or a membrane separator such as “KM-8100N” available from Kuraray Co, Ltd.

The plasma introducing circuit 3 extends from the plasma inlet 2 to a plasma intake port 9 of a plasma component adsorbent unit 8, which is a sort of a blood purifying unit, through a plasma inlet drip chamber 4, then through a plasma feed pump 5 and finally through a plasma purifier drip chamber 6. The plasma adsorbing system also include a plasma return fluid circuit 19 extending from a plasma outlet port 10 of the plasma component adsorbent unit 8 to a plasma return outlet 20 through a heater 17 and then through a plasma return drip chamber 18. A bypass fluid circuit 22 for bypassing the plasma feed pump 5 and the plasma component adsorbent unit 8 is disposed between a portion of the plasma introducing fluid circuit 3, which extends between the plasma inlet drip chamber 4 and the plasma feed pump 5, and a portion of the plasma return fluid circuit 19 which extends between the heater 17 and the plasma return drip chamber 18 This bypass fluid circuit 22 has a circuit switching valve 21 disposed thereon. In this way, an extracorporeal circulating circuit is thus completed.

Also, for detecting the pressure at various portions of the extracorporeal circulating circuit, the plasma adsorbing system also includes a plasma inlet pressure gauge 23 fluid-connected with the plasma inlet drip chamber 4 for measuring the pressure at an intake port of the feed pump 5, a plasma purifier pressure gauge 24 fluid-connected with the plasma purifier drip chamber 6 for detecting the pressure at a discharge port of the feed pump 5, and a plasma return pressure gauge 25 fluid connected with the return plasma drip chamber 18 for measuring the pressure in the plasma return fluid circuit 19.

The plasma component adsorbent unit 8 referred to above is of a type having a mass of adsorbent material filled in a column and is chosen to suit to a particular type of materies morbi that is desired to be removed. While any of various adsorbing methods such as, for example, a physical adsorption, a chemical adsorption and an affinity adsorption are available depending on the characteristics of the adsorbent material, selection of one of those various adsorbing methods employed in the plasma component adsorbent unit 8 is preferably made with a view to the use of a particular adsorbent material capable of exhibiting an excellent adsorbing performance with respect to target materials desired to be removed and being non-specific to useful material. The adsorbent material to be used in the plasma component adsorbent unit 8 may be made up of a mass of beads or fibers.

As the separated plasma is supplied from the plasma separating unit 1, the plasma inlet pressure increases. This plasma inlet pressure is measured by the plasma inlet pressure gauge 23 at all times and monitored by a control unit 26 including a pump control circuit 27 built therein for automatically controlling the flow of the plasma feed pump 5 so that the measured pressure can fall within a predetermined range with respect to a preset pressure.

By measuring the plasma inlet pressure accurately and controlling the flow of the plasma feed pump 5 so that the plasma inlet pressure can fall within the predetermined range with respect to the preset pressure, it is possible to process the plasma at a flow rate consistent with change in the amount of the separated plasma supplied from the plasma separating unit 1. In the practice of the present invention, it is preferred that the measured pressure be controlled to be within the range of ±5 mmHg and more preferably ±2 mmHg with respect to the preset pressure. At the same time the flow of the plasma discharged from the plasma feed pump 5 is preferably within the range of ±5 ml/min, and more preferably ±3 ml/min, with respect to the flow of the plasma from the plasma separating unit 1.

The flow of the plasma discharged from the plasma feed pump 5 is to be determined by the control unit 26 in reference to the number of revolutions of the plasma feed pump 5 and the pressure measured by the plasma purifier pressure gauge 24, whereas the flow of the plasma emerging from the blood separating unit 1 is determined by the control unit 26 in reference to the number of revolutions of the plasma feed pump 5 and the pressure measured by the plasma inlet pressure gauge 23.

Even the plasma purifier pressure and the plasma return pressure are monitored by the control unit 26 through the plasma purifier pressure gauge 24 and the plasma return pressure gauge 25, respectively, so that in the event that each of those pressures exceeds a respective predetermined value, an abnormality detecting circuit 28 built in the control unit 26 detects such event with the pump control circuit 27 in the control unit 26 consequently causing the plasma feed pump 5 to halt. At the same time, a valve control circuit 29 built in the control unit 26 causes the valve 21 in the bypass circuit to open Accordingly, with no need to interrupt the flow of the separated plasma from the plasma separating unit 1, the system can deal with such an abnormality Also, in the event that the plasma feed pump 5 is halted by reason of any other system malfunction and/or an erroneous manual operation, the valve 21 in the bypass circuit 22 can be opened by the control unit 26 and, accordingly, it is possible to avoid the extracorporeal circulating circuit from being closed In other words, the bypass circuit 22 is opened when the system is brought in an alert condition as a result of occurrence of an abnormality or malfunction.

In view of the foregoing, the blood processing system of the present invention is preferably equipped with any suitable warning device capable of providing an audio and/or video warning indication, such as a blinking lamp and/or an alarm, to the attendant operator in the event that the valve 21 in the bypass circuit 22 is opened

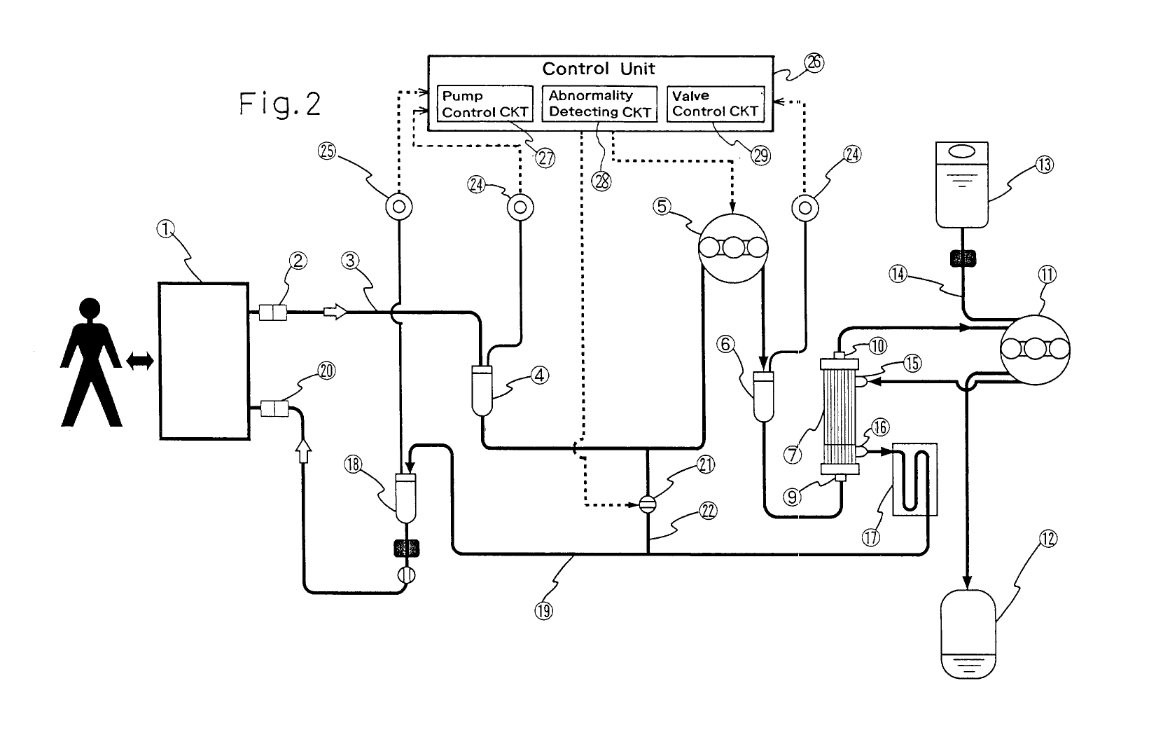

Referring now to FIG. 2 showing a blood processing system according to another preferred embodiment of the present invention, the illustrated blood processing system is a plasma purifying and exchange transfusing system. The plasma purifying unit employed in the system shown in FIG. 2 is a membrane module 7, and the plasma introducing fluid circuit 3 extending from the plasma separating unit 1 is fluid-connected with a plasma intake port 9 of this membrane module 7 in a manner similar to that with the adsorbent unit 8 shown in FIG. 1 The membrane module 7 is operable to filter the plasma and has a filtrate discharge port 10 from which a liquid component separated from the plasma is discharged and a plasma outlet port 16 from which the plasma having been so purified emerged into the plasma return fluid circuit 19 through the heater 17. A filtrate discharge circuit fluid-connected with the filtrate discharge port 10 of the membrane module 7 is fluid-connected with a filtrate disposal container 12 through a filtrate feed pump 11

The filtrate feed pump 11 is also fluid connected with an upstream portion of a supplementary liquid supply circuit 14 that extends from a supplementary liquid reservoir 13, while a downstream portion of the supplementary liquid supply circuit 14 that extends from the filtrate feed pump 11 is fluid-connected with a supplementary liquid feed port 15 of the membrane module 7. The supplementary liquid feed circuit 14 is used so that a supplementary liquid from the supplementary liquid reservoir 13 can be supplied into the membrane module 7 and be subsequently mixed with the plasma in a quantity corresponding to the quantity of the filtrate separated from the plasma. This supplementary liquid is generally employed in the form of a physiologically compatible fluid substitute.

The plasma return fluid circuit 19 extends from the plasma outlet port 16 of the membrane module 7, from which the purified plasma emerges, to the plasma return port 20 in a manner similar to that shown in FIG. 1.

Similar to the previously described embodiment, the bypass fluid circuit 22 for bypassing the plasma feed pump 5 and the plasma purifying and exchange transfusing unit 7 is disposed between the portion of the plasma introducing fluid circuit 13, which extends between the plasma inlet drip chamber 4 and the plasma feed pump 5, and the portion of the plasma return fluid circuit 19 which extends between the heater 17 and the plasma return drip chamber 18 This bypass fluid circuit 22 has the circuit switching valve 21 disposed thereon, which when closed establishes a bypass circuit Also, the plasma adsorbing system also includes the plasma inlet pressure gauge 23 fluid-connected with the plasma inlet drip chamber 4, the plasma purifier pressure gauge 24 fluid-connected with the plasma purifier drip chamber 6, and the plasma return pressure gauge 25 fluid-connected with the return plasma drip chamber 18

The membrane module 7 referred to above is of a type having a plasma component fractionating membrane built therein, which membrane is preferably in the form of a hollow fiber or a plain membrane. The plasma component fractionating membrane is operable to separate the plasma component selectively into a high molecular component and a low molecular component, and the molecular weight to be fractionated can be set to any desired value depending on the molecular weight of the target materies morbi that is desired to be fractionated. Also, for the plasma component fractionating membrane, a homogeneous micropored membrane, a microfiltration membrane or a so-called asymmetric structural membrane made up of a porous support layer and a micropored structural layer can be generally employed. Material for this membrane may include a polyvinyl alcohol (PVA) polymer, ethylene vinyl alcohol (EVA) polymer, a cellulose derivative such as, for example, cellulose diacetate, or polypropylene.

Although the present invention has been fully described in connection with the preferred embodiments thereof with reference to the accompanying drawings which are used only for the purpose of illustration, those skilled in the art will readily conceive numerous changes and modifications within the framework of obviousness upon the reading of the specification herein presented of the present invention. Accordingly, such changes and modifications are, unless they depart from the scope of the present invention as delivered from the claims annexed hereto, to be construed as included therein.

What is claimed is:

1. A blood processing system for purifying a plasma in a blood which comprises.
a plasma separating unit for separating a plasma from a blood,
a plasma purifying unit for purifying the separated plasma,
a plasma introducing fluid circuit for introducing the separated plasma into the plasma purifying unit;
a plasma feed pump disposed in the plasma introducing fluid circuit and having a plasma intake port and a plasma discharge port;
a plasma inlet pressure gauge for measuring a pressure of the plasma at the plasma intake port of the plasma feed pump;
a plasma return fluid circuit for returning the plasma, which has been purified by the plasma purifying unit, back to the plasma separating unit;
a bypass fluid circuit extending between the plasma introducing fluid circuit and the plasma return fluid circuit for bypassing the plasma feed pump and the plasma purifying unit;
a valve disposed in the bypass circuit for opening the bypass circuit in the event of an occurrence of operative abnormality in the blood purifying system; and
a control unit for controlling the plasma feed pump in reference to the plasma inlet pressure measured by the plasma inlet pressure gauge to render the flow of the plasma inlet pressure to fall within a predetermined range with respect to a preset pressure.

2. The blood processing system as claimed in claim 1, wherein the control unit controls the plasma feed pump to render the plasma inlet pressure to fall within the range of ±5 mmHg with respect to the preset pressure.

3. The blood processing system as claimed in claim 1, wherein the control unit controls the plasma feed pump to render the plasma discharged from the plasma feed pump to be within a range of ±5 ml/min. with respect to the flow of the plasma from the plasma separating unit.

4. The blood processing system as claimed in claim 1, further comprising a pressure gauge disposed between the plasma feed pump and the plasma purifying unit for detecting a plasma purifier pressure and wherein in the event that the plasma purifier pressure exceeds a predetermined value, the control unit halts the plasma feed pump and causes the valve on the bypass fluid circuit to open.

5. The blood processing system as claimed in claim 1, further comprising a pressure gauge disposed in the plasma return fluid circuit for detecting a pressure of the plasma being returned and wherein in the event that the pressure of the plasma being returned exceeds a predetermined value the control unit halts the plasma feed pump and causes the valve on the bypass fluid circuit to open.

6. The blood processing system as claimed in claim 1, wherein in the event that the plasma feed pump is halted, the control unit causes the valve on the bypass fluid circuit to open.

7. The blood processing system as claimed in claim 1, wherein the plasma purifying unit is selected from the group consisting of a plasma component fractionating membrane module and a plasma component adsorbent unit

8. The blood processing system as claimed in claim 7, wherein the plasma purifying unit comprises the plasma component fractionating membrane module, and further comprising a filtrate feed pump operable in association with the plasma feed pump for draining a filtrate separated from the plasma by the plasma component fractionating membrane module.

9. The blood processing system as claimed in claim 8, further comprising a supplementary liquid supply circuit for supplying a supplementary liquid to the plasma component fractionating membrane module by means of the filtrate feed pump to thereby supplement a filtrated plasma with the supplementary liquid in a quantity corresponding to the quantity of the filtrate separated from the plasma.
